(19) United States
(12) Patent Application Publication
(10) Pub. No.: US 2014/0046357 A1
(43) Pub. Date: Feb. 13, 2014

(54) DILATION DEVICE

(71) Applicant: COOK MEDICAL TECHNOLOGIES LLC, Bloomington, IN (US)

(72) Inventor: Wen Hong NEOH, Bloomington, IN (US)

(73) Assignee: COOK MEDICAL TECHNOLOGIES LLC, Bloomington, IN (US)

(21) Appl. No.: 13/961,425

(22) Filed: Aug. 7, 2013

Related U.S. Application Data
(60) Provisional application No. 61/681,164, filed on Aug. 9, 2012.

(51) Int. Cl. A61M 29/00

(52) U.S. Cl. CPC A61M 29/00
USPC 606/191

(57) ABSTRACT

A dilator for enlarging an opening through a body tissue may include a shaft portion extending along a longitudinal axis of the dilator and including a proximal end and a distal end. The dilator may include a dilator tip positioned at the distal end of the shaft portion and including a proximal end, a distal end, and an outer surface extending between the proximal and distal ends of the dilator tip. The outer surface may include a first diameter at the proximal end of the dilator tip and a second diameter at the distal end of the dilator tip. The first diameter may be larger than the second diameter. The outer surface may include a concave taper between the first diameter and the second diameter. The dilator tip may include a plurality of projections and/or recesses extending away from the outer surface of the dilator tip.

### CROSS-REFERENCE TO RELATED APPLICATIONS

This application claims priority and the benefit of U.S. Provisional Patent Application Ser. No. 61/681,164, filed Aug. 9, 2012, which is incorporated herein by reference in its entirety.

### TECHNICAL FIELD

The present disclosure relates generally to medical devices. More particularly, it relates to a dilation device, such as a dilator for enlarging an opening through a body tissue.

### BACKGROUND

Various medical procedures involve gaining percutaneous access into a patient. For example, a medical device may be inserted percutaneously into a patient to access the patient's gastrointestinal tract for placement of a G-tube, to access the patient's trachea as part of a tracheostomy, or to access the patient's vascular system as part of an aortic intervention procedure. To minimize trauma to the patient, it has been found desirable to initially incise a small opening through the patient's tissue as an entrance and then to enlarge the opening with further dilation. In many situations, a series of dilators of increasing diameter are used to gradually expand the opening. In such a procedure, a hollow needle may be inserted through the tissue and into the patient. A guide wire may be inserted through the needle, and the needle may be withdrawn over the guide wire and removed, leaving the guide wire in place within the opening. A dilator with a tapered tip may be advanced over the guide wire and into the opening to enlarge the opening. The dilator may be withdrawn and removed, and a second dilator having a tapered tip with a larger diameter may be advanced over the guide wire to further enlarge the opening. These steps may be repeated using dilators having increasingly larger tapered tips until the opening is expanded to the desired size.

A single dilator having a steeper taper may be used instead of the series of increasingly larger dilators to expand the opening. The use of a single dilator may be advantageous because it reduces the number of steps and the number of components involved in dilating the opening. However, the larger diameter of the single dilator relative to the opening and the greater contact area between the surface of the dilator and the patient's tissue may make it difficult for the physician to push the dilator through the opening. The increased resistance to pushing the dilator through the opening may cause discomfort to the patient during the dilation procedure.

### SUMMARY

The present embodiments provide a dilation device, which may be used for enlarging an opening through a body tissue.

In one example, a dilator for enlarging an opening through a body tissue may include a shaft portion extending along a longitudinal axis of the dilator. The shaft portion may include a proximal end and a distal end. The dilator may include a dilator tip positioned at the distal end of the shaft portion. The dilator tip may include a proximal end, a distal end, and an outer surface extending between the proximal end and the distal end of the dilator tip. The outer surface may include a first diameter at the proximal end of the dilator tip and a second diameter at the distal end of the dilator tip. The first diameter may be larger than the second diameter. The outer surface may include a concave taper between the first diameter and the second diameter. The dilator tip may include a plurality of projections and/or recesses extending away from the outer surface of the dilator tip.

In another example, a dilator for enlarging an opening through a body tissue may include a shaft portion and a dilator tip. The shaft portion may extend along a longitudinal axis of the dilator. The shaft portion may include a proximal end and a distal end. The dilator tip may be positioned at the distal end of the shaft portion. The dilator tip may include a proximal end, a distal end, and an outer surface extending between the proximal end and the distal end of the dilator tip. The outer surface may include a proximal region positioned near the proximal end of the dilator tip and a distal region positioned near the distal end of the dilator tip. A diameter of the outer surface may decrease in a proximal to distal longitudinal direction. The proximal region of the outer surface may include a taper that is greater than a taper of the distal region of the outer surface.

In another example, a method of enlarging an opening in a body tissue may include introducing a distal end of a dilator tip into the opening. The dilator tip may include an outer surface. The outer surface may include an increasing diameter in a distal to proximal longitudinal direction. The diameter may increase in a concave taper. The method may include advancing the dilator tip into the opening to enlarge the opening with the increasing diameter of the dilator tip. The method may include manipulating a portion of the body tissue surrounding the opening with a plurality of projections extending away from the outer surface of the dilator tip.

Other systems, methods, features, and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTIONS OF THE DRAWINGS

FIG. 1 shows a distal portion of one example of a dilation device including a plurality of projections arranged in one exemplary configuration.

FIG. 2 shows a longitudinal cross-sectional view of the dilation device of FIG. 1.

FIG. 3 shows a distal portion of another example of a dilation device including a plurality of projections arranged in another exemplary configuration.

FIG. 4 shows a distal portion of another example of a dilation device including a plurality of projections arranged in another exemplary configuration.

FIG. 5 shows a distal portion of another example of a dilation device including a plurality of indentations arranged in one exemplary configuration.

FIG. 6 shows a distal portion of another example of a dilation device including a plurality of indentations arranged in another exemplary configuration.

FIG. 7 shows a distal portion of another example of a dilation device including a groove arranged in one exemplary configuration.

FIG. 8 shows a distal portion of another example of a dilation device including a plurality of grooves arranged in another exemplary configuration.

### DETAILED DESCRIPTION OF THE DRAWINGS AND THE PRESENTLY PREFERRED EMBODIMENTS

In the present disclosure, the term “proximal” refers to a direction that is generally toward a physician during a medical procedure, while the term “distal” refers to a direction that is generally toward a target site within a patient's anatomy during a medical procedure.

Various medical devices for introduction into a body of a patient are described herein. Preferred embodiments relate to a dilation device, which may be useful for gaining percutaneous access into a patient's body. However, this disclosure is not so limited, and the dilation device may be used in other portions of the human or mammal anatomy to enlarge an opening through any type of body tissue.

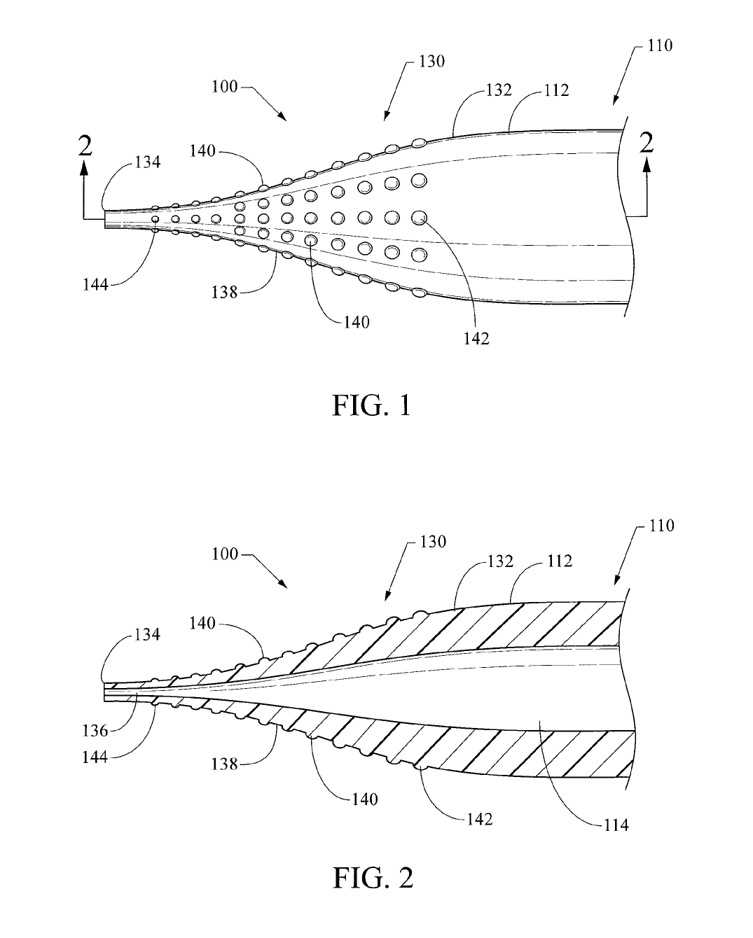

FIG. 1 illustrates a distal portion of one example of a dilator 100, and FIG. 2 is a longitudinal cross sectional view of the distal portion of the dilator illustrated in FIG. 1. The dilator 100 may include a shaft portion 110 and a dilator tip 130 positioned at the distal end of the dilator. The shaft portion 110 may include an elongate member defined around a longitudinal axis and having a proximal end (not shown) and a distal end 112. In one example, the shaft portion 110 may be configured as a tubular member. To that end, the shaft portion 110 may include a lumen 114 extending longitudinally within the shaft portion. In one example, the shaft portion 110 may have a substantially cylindrical shape. In other words, a transverse cross section of the shaft portion 110 may have a circular shape. In other examples, the shaft portion 110 may be configured as an elongate member having a cross section with any other shape such as, for example, elliptical, triangular, rectangular, or any other polygonal or non-polygonal shape. The shaft portion 110 may include an outer surface which may be smooth (i.e., free of surface textures). Alternatively, the outer surface of the shaft portion 110 may be textured.

The dilator tip 130 may extend distally from the distal end 112 of the shaft portion 110. The dilator tip 130 may be separate from or integral with the shaft portion 110 as further described below. The dilator tip 130 may be defined about the longitudinal axis such that the shaft portion 110 and the dilator tip 130 are coaxial. The dilator tip 130 may have a proximal end 132 and a distal end 134. The proximal end 132 of the dilator tip 130 may be attached to the distal end 112 of the shaft portion 110. In one example, the dilator tip 130 may have a length (i.e., a distance between the proximal end 132 and the distal end 134) of between about 0.25 in and about 2.25 in, preferably between about 0.5 in and about 1.5 in. In other examples, the dilator tip 130 may have any other suitable length.

The dilator tip 130 may include a lumen 136 extending longitudinally within the dilator tip. The lumen 136 of the dilator tip may be in fluid communication with the lumen 114 of the shaft portion 110. The lumen 114 of the shaft portion 110 and the lumen 136 of the dilator tip 130 may collectively define a main lumen of the dilator 100. The main lumen of the dilator 100 may extend longitudinally within the dilator, and may be in fluid communication with a proximal end opening at the proximal end of the shaft portion 110 and a distal end opening at the distal end 134 of the dilator tip 130. In one example, the distal end opening may have a diameter of between about 0.0175 in and about 0.057 in, preferably between about 0.035 in and about 0.038 in. In other examples, the distal end opening may have any other suitable diameter. The main lumen of the dilator 100 may have a diameter that is substantially the same as the diameter of the distal end opening. In other words, the main lumen of the dilator 100 may have a substantially constant diameter along the length of the dilator. In other examples, the diameter of the main lumen may vary longitudinally along the dilator 100. The main lumen of the dilator 100 may be configured to receive a guide wire to aid in advancing the dilator into an opening in a body tissue as further described below.

The dilator tip 130 may include an outer surface 138 extending between the proximal end 132 and the distal end 134 of the dilator tip. In one example, the dilator tip 130, or a portion thereof, may be tapered. To that end, the proximal end 132 of the dilator tip 130 may have a first diameter, and the distal end 134 of the dilator tip may have a second diameter, which may be different than the first diameter. In one example, the first diameter of the proximal end 134 may be greater than the second diameter of the distal end 134. In other words, the proximal end 132 of the dilator tip 130 may have a larger diameter than the distal end 134 of the dilator tip. In one example, the proximal end 132 of the dilator tip 130 may have a diameter between about 7 Fr and about 36 Fr, preferably between about 14 Fr and about 24 Fr. Additionally, or alternatively, the distal end 134 of the dilator tip 130 may have a diameter between about 0.0325 in and about 0.087 in, preferably between about 0.05 in and about 0.068 in. In other examples, the proximal end 132 and the distal end 134 may have any suitable diameters. The diameter of the outer surface 138 of the dilator tip 130 may change in a longitudinal direction between the proximal end 132 and the distal end 134 of the dilator tip 130. For example, the diameter of the outer surface 138 may decrease in a proximal to distal direction from the proximal end 132 to the distal end 134. In other words, the outer surface 138 of the dilator tip 130 may be tapered between the proximal end 132 and the distal end 134. The tapered outer surface 138 may aid in passing the dilator tip 130 through an opening in a body tissue to enlarge the opening as further described below.

A plane segment may include the longitudinal axis of the dilator tip 130 and extend radially outward away from the longitudinal axis. The plane segment may intersect the outer surface 138 of the dilator tip 130. In one example, the outer surface 138 may have a straight taper. In this example, the intersection of the plane segment and the outer surface 138 may be a substantially straight line extending between the proximal end 132 and the distal end 134 of the dilator tip 130. In other words, the outer surface 138 of the dilator tip 130 may have a generally frustoconical shape with substantially straight sides.

In another example, the outer surface 138 may have a nonlinear taper as shown in FIGS. 1-2. In this example, the intersection of the plane segment and the outer surface 138 may be a nonlinear curve extending between the proximal end 132 and the distal end 134 of the dilator tip 130. In other words, the outer surface 138 of the dilator tip 130 may have a generally frustoconical shape with curved sides (e.g., a funnel shape). In one example, the nonlinear taper may be a concave taper as shown in FIGS. 1-2. In this example, the outer surface 138 may be curved inward toward the longitudinal axis of the dilator tip 130. In other words, the outer surface 138 may be configured as a contoured outer surface. The contoured outer surface 138 may have a radius of curvature, which may depend on the length of the dilator tip 130 (e.g., the distance between the proximal end 132 and the distal end 134). The contoured outer surface 138 may have any suitable radius of curvature.

A proximal region of the outer surface 138 near the proximal end 132 of the dilator tip 130 may have a steeper taper than a distal region of the dilator tip near the distal end 134. In other words, a line coplanar with the longitudinal axis and tangent to the proximal region of the outer surface may form a greater angle with the longitudinal axis (i.e., may have a greater slope) than another line coplanar with the longitudinal axis and tangent to the distal region of the outer surface. The taper of the outer surface 138 may become increasingly less steep (i.e., increasingly flatter) in a proximal to distal direction between the proximal end 132 and the distal end 134. In other words, the outer surface 138 of the dilator tip 130 may be generally funnel-shaped as shown in FIGS. 1-2. In one example, the proximal region of the outer surface 138 near the proximal end 132 of the dilator tip 130 may have a taper of between about 2.4° and about 3.6°. In other words, the diameter of the outer surface 138 of the dilator tip 130 may decrease by between about 0.084 mm and about 0.126 mm per mm of length at the proximal region of the outer surface. Additionally, or alternatively, the distal region of the outer surface 138 near the distal end 134 of the dilator tip 130 may have a taper of between about 1.2° and about 2.4°. In other words, the diameter of the outer surface 138 of the dilator tip 130 may decrease by between about 0.042 mm and about 0.084 mm per mm of length at the distal region of the outer surface. In other examples, the outer surface 138 may have any other taper. The concave taper may aid in passing the dilator tip 130 through an opening in a body tissue to enlarge the opening as further described below. In another example, the nonlinear taper may be a convex taper in which the outer surface 138 may be curved outward away from the longitudinal axis of the dilator tip 130.

In some examples, the dilator tip 130 may include one or more surface features on the outer surface 138 to form a textured outer surface. In one example, the surface features may be configured as projections or bumps 140 extending outward away from the outer surface 138 as shown in FIGS. 1-2. Each projection 140 may include a base at the outer surface 138 and a body extending from the base. The body may extend outward from the base in a direction away from the longitudinal axis of the dilator tip 130. The base may be substantially circular as shown in FIGS. 1-2. In other examples, the base may have any shape such as, for example, elliptical, triangular, rectangular, or any other polygonal or non-polygonal shape. The body may be configured as a protrusion, such as a dome, extending from the base. Preferably, the body may be substantially smooth or substantially free of sharp edges which may cause pain and/or discomfort to the patient upon passing the dilator tip 130 through an opening in a body tissue as further described below. In other words, preferably, the body may be substantially atraumatic. To that end, the body may have a generally hemispherical shape as shown in FIGS. 1-2. In other examples, the body may have any shape such as, for example, an ellipsoid, a pyramid, a prism, or any other three-dimensional geometric shape. The projections 140 may have any suitable size. An aspect ratio of the projections 140 may be a ratio of the height of the projections to the diameter of the base of the projections. In one example, an aspect ratio of the projections 140 may be between about 1:1 and about 1:3. Additionally, or alternatively, the projections 140 may have a height of less than or equal to about 1 mm. In other examples, the projections may have any other suitable diameter and/or height.

The projections 140 may be configured to decrease the resistance to passing the dilator tip 130 through the opening in the body tissue by manipulating the tissue as further described below. To that end, the projections 140 may be arranged on the outer surface 138 of the dilator tip 130 in any desired pattern. In one example, the projections 140 may be arranged in a series of substantially straight rows as shown in FIGS. 1-2. The rows of projections may be spaced from one another around the circumference of the dilator tip 130, and each row of projections may extend generally longitudinally along the dilator tip 130 in a substantially straight line.

Each projection 140 in a row of projections may be substantially the same size. Alternatively, a row of projections 140 may include projections having different sizes. In one example, a row of projections 140 may include a proximal projection 142 positioned near the proximal end 132 of the dilator tip 130 and a distal projection 144 positioned near the distal end 134 of the dilator tip. The proximal projection 142 may be larger than the distal projection 144. For example, the base of the proximal projection 142 may have a larger diameter than the base of the distal projection 144. In other words, the base of the proximal projection 142 may be wider and/or longer than the base of the distal projection 144. Additionally, or alternatively, the body of the proximal projection 142 may have a larger height than the body of the distal projection 144. In other words, the proximal projection 142 may extend farther from the outer surface 138 of the dilator tip 130 than the distal projection 144. The size of each projection 140 in the row of projections may vary from the proximal projection 142 to the distal projection 144. For example, the proximal projection 142 may be the largest projection in the row, and an adjacent projection distal of the proximal projection 142 may be smaller than the proximal projection 142. In one example, each projection 140 of the row of projections may be larger than the adjacent projection distal of the projection. In other words, each successive projection in the proximal to distal direction may be smaller than each of the preceding projections. Accordingly, the distal projection 144 may be the smallest projection in the row of projections. In other examples, the projections may be increasingly larger in the proximal to distal direction, or the size of the projections may vary randomly along the row of projections.

The size of each projection 140 may be related to the location of the projection on the distal tip 130. For example, the size of each projection 140 may be related to the longitudinal position of the projection along the distal tip 130. One projection (e.g., the proximal projection 142) may be disposed at a first longitudinal position, and another projection (e.g., the distal projection 144) may be disposed at a second longitudinal position distal of the first longitudinal position. The distal tip 130 may be tapered such that the diameter of the distal tip at the first longitudinal position may be greater than the diameter of the distal tip at the second longitudinal position as described above. The projections may be sized such that the size of the proximal projection 142 relative to the diameter of the distal tip 130 at the first longitudinal position is substantially the same as the size of the distal projection 144 relative to the diameter of the distal tip at the second longitudinal position. In other words, the ratio of the diameter and/or height of the proximal projection 142 to the diameter of the distal tip 130 at the first longitudinal position may be substantially the same as the ratio of the diameter and/or height of the distal projection 144 to the diameter of the distal tip at the second longitudinal position. In other examples, the projections may be sized such that the size of the proximal projection 142 relative to the diameter of the distal tip 130 at the first longitudinal position is greater than or less than the size of the distal projection 144 relative to the diameter of the distal tip at the second longitudinal position.

Additionally, or alternatively, the width of the space between adjacent rows of projections 140 around the circumference of the dilator tip 130 may be related to the longitudinal position along the dilator tip. For example, the circumferential space between adjacent rows of projections 140 at the longitudinal position of the proximal projection 142 may be larger than the circumferential space between the adjacent rows of projections at the longitudinal position of the distal projection 144. In one example, the ratio of the width of the space to the diameter of the distal tip 130 at the longitudinal position may be substantially constant. In other examples, the ratio of the width of the space to the diameter of the distal tip 130 may increase or decrease along the length of the dilator tip.

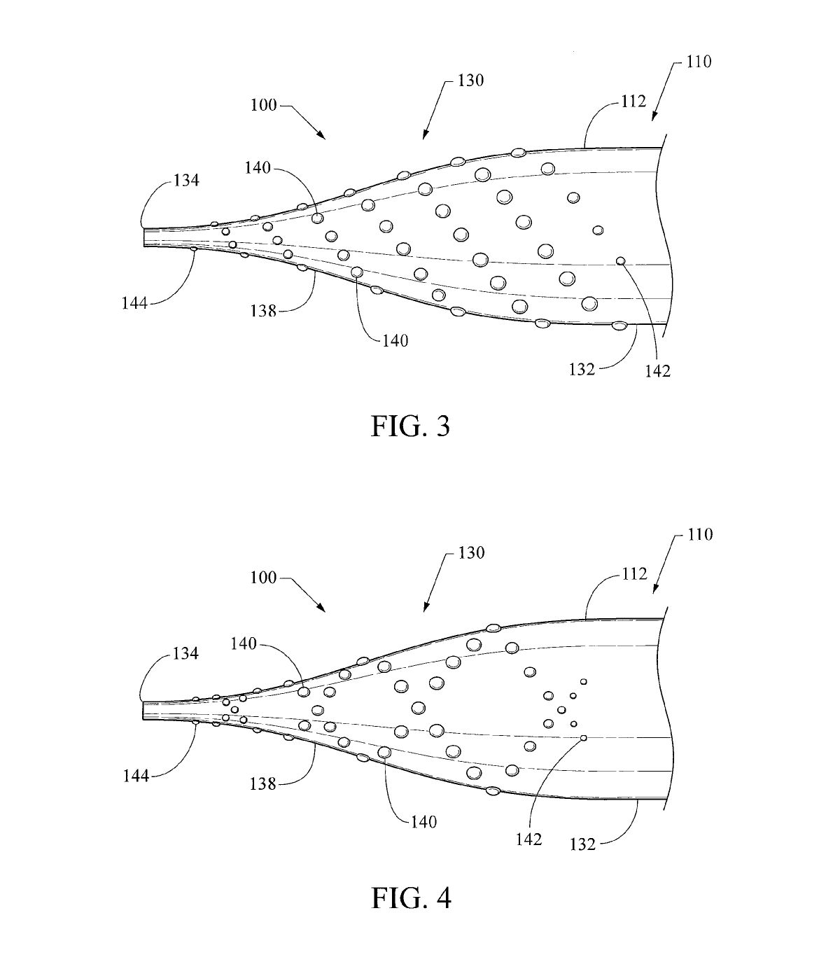

In another example, the projections 140 may be arranged in a spiral pattern as shown in FIGS. 3-4. FIG. 3 depicts one example of the projections 140 arranged in a single spiral pattern. In this example, the projections 140 may be arranged in a spiral or helical pattern extending longitudinally and circumferentially along the distal tip 130. The spiral pattern may include a plurality of adjacent turns, which may be angled with respect to a right circumference (i.e., a circumference perpendicular to the longitudinal axis) of the dilator tip 130. Each turn of the spiral pattern may extend about 360 degrees in a circumferential direction around the outer surface 138 of the dilator tip 130. The longitudinal distance between adjacent turns of the spiral pattern may be referred to as the pitch of the spiral pattern. The spiral pattern may have any suitable pitch. In one example, the pitch of the spiral pattern may remain substantially constant between the proximal end 132 and the distal end 134 of the dilator tip 130. In other examples, the pitch may increase or decrease in a proximal to distal longitudinal direction between the proximal end 132 and the distal end 134.

The spiral pattern may extend along the distal tip 130 in a right-handed or a left-handed configuration. In the right-handed configuration, the spiral pattern may extend longitudinally in a distal to proximal direction and circumferentially in a clockwise direction when viewing the distal end 134 of the dilator tip 130 head on as shown in FIG. 3. In other words, the spiral pattern may resemble the threads of a screw disposed on the outer surface 138 of the dilator tip 130. In the left-handed configuration, the spiral pattern may extend longitudinally in a distal to proximal direction and circumferentially in a counterclockwise direction when viewing the distal end 134 of the dilator tip 130 head on. In other words, the spiral pattern may resemble the threads of a reverse-threaded screw disposed on the outer surface 138 of the dilator tip 130. In either the right-handed or the left-handed configuration, such a pattern may aid in passing the dilator tip 130 through an opening in a body tissue by twisting the dilator tip as further described below.

FIG. 4 depicts one example of the projections 140 arranged in a double-spiral pattern. In this example, the projections 140 may include two series of projections, each arranged in a spiral or helical pattern extending longitudinally and circumferentially along the distal tip 130. The spiral pattern of the first series of projections 140 may extend along the distal tip 130 in a right-handed configuration as described above, and the spiral pattern of the second series of projections may extend along the distal tip in a left-handed configuration also as described above. The double-spiral pattern may resemble a double helix disposed on the outer surface 138 of the dilator tip 130. Such a pattern may aid in passing the dilator tip 130 through an opening in a body tissue by twisting the dilator tip in one or more directions as further described below.

In other examples, the projections 140 may be arranged in any suitable pattern (e.g., random). Preferably, the projections 140 may be disposed at varying longitudinal and circumferential positions about the outer surface 138 of the dilator tip 130 to manipulate a body tissue upon passage of the dilator tip through an opening in the body tissue as further described below.

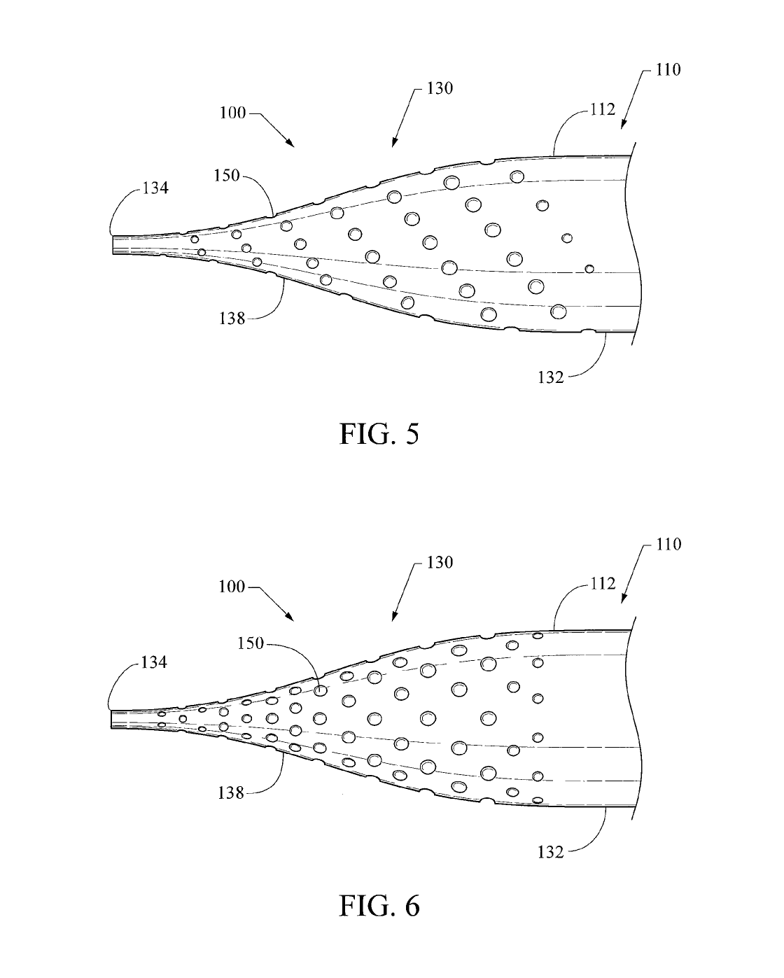

In one example, the surface features may be configured as indentations or dimples 150 in the outer surface 138 as shown in FIGS. 5-6. Each indentation 150 may be configured as a depression in the outer surface 138 extending inward toward the longitudinal axis of the dilator tip 130. Each indentation 150 may include an opening in the outer surface 138 and a void extending inward from the opening. The opening may have any size or shape. For example, the opening may have a size and/or shape similar to that of the base of the projection 140 described above. Similarly, the void may have any size or shape such as, for example, a size and/or shape similar to that of the body of the projection 140 described above. Additionally, or alternatively, the indentations 150 may be arranged on the outer surface 138 of the dilator tip 130 in any suitable pattern as described above with reference to the projections 140. For example, the indentations 150 may be arranged in a series of substantially straight rows, in a spiral pattern as shown in FIG. 5, in a random pattern as shown in FIG. 6, or in any other pattern. Preferably, the indentations 150 may be disposed at varying longitudinal and circumferential positions about the outer surface 138 of the dilator tip 130 as described above with reference to the projections 140. The indentations 150 may reduce the surface area of the distal tip 130 which may engage a body tissue upon passing the dilator tip through an opening in the body tissue as further described below. The reduced contact area between the dilator tip 130 and the body tissue may reduce the force required to pass the dilator tip through the opening. Additionally, or alternatively, the reduced contact area between the dilator tip 130 and the body tissue may reduce the pain and/or discomfort which may be experienced by the patient during introduction of the dilator tip through the opening in the body tissue.

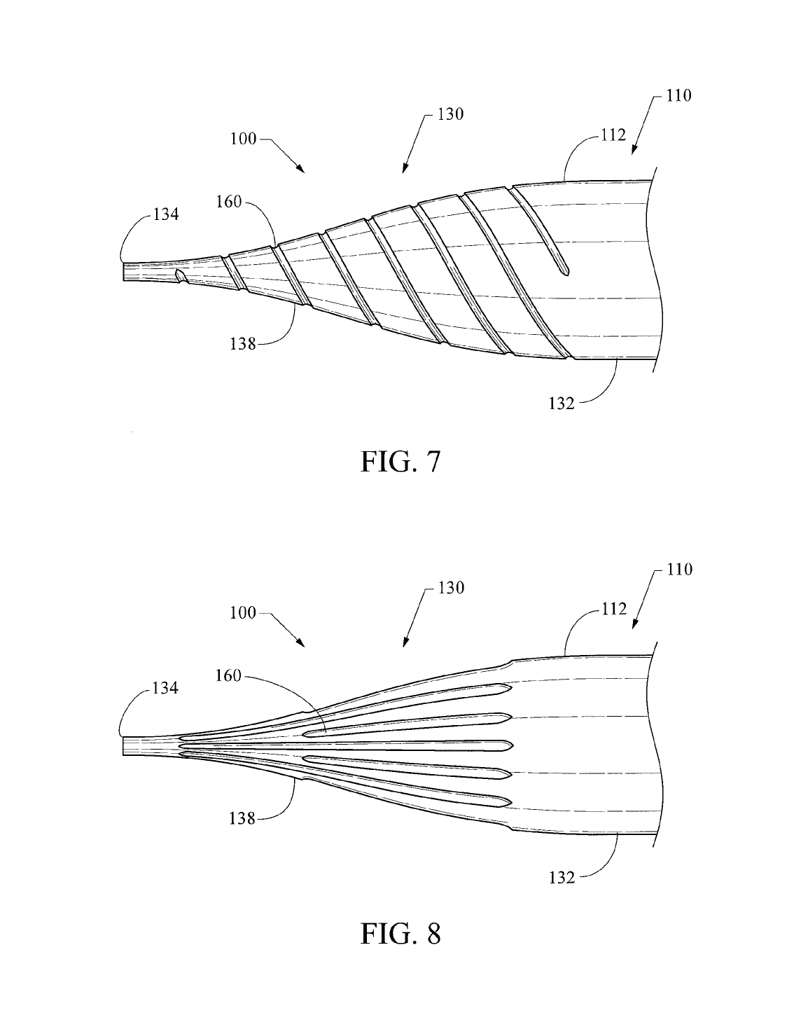

In another example, the surface features may be configured as one or more grooves or furrows 160 in the outer surface 138 as shown in FIGS. 7-8. Each groove 160 may be configured as a channel in the outer surface 138 extending inward toward the longitudinal axis of the dilator tip 130. The groove 160 may have any suitable size and/or shape. For example, the groove 160 may have a semicircular cross sectional shape as shown in FIG. 7. In other examples, the groove 160 may have a triangular, rectangular, or any other cross sectional shape. The groove 160 may include an opening positioned on the outer surface 138 of the dilator tip. The opening may have any suitable width. For example, the width of the opening may have a size that is similar to the diameter of the base of the projection 140 described above. Additionally, or alternatively, the groove 160 may extend along the outer surface 138 of the dilator tip 130 in any suitable pattern as described above with reference to the projections 140. For example, the groove 160 may extend longitudinally and circumferentially along the dilator tip 130 in a spiral pattern as shown in FIG. 7. In other examples, the dilator tip 130 may include multiple grooves 160 each having a spiral pattern (e.g., a double-spiral or double-helix pattern). In another example, the groove 160 may be configured as a series of substantially straight grooves extending longitudinally along the dilator tip 130 as shown in FIG. 8. The grooves 160 may reduce the surface area of the distal tip 130 which may engage a body tissue upon passing the dilator tip through an opening in the body tissue as further described below. The reduced contact area between the dilator tip 130 and the body tissue may reduce the force required to pass the dilator tip through the opening and/or reduce the pain and/or discomfort which may be experienced by the patient during introduction of the dilator tip through the opening in the body tissue.

In any of the examples described herein, the dilator 100 may be made from any suitable material known in the art including, for example, a polymer or a metal. The shaft portion 110 and the dilator tip 130 may be made from the same or a different material. Suitable materials for the shaft portion 110 and/or the dilator tip 130 may include, for example, high density polyethylene (HDPE), polypropylene, silicone, polyurethane, polytetrafluoroethylene (PTFE), expanded polytetrafluoroethylene (ePTFE), polyamide, polyvinyl chloride (PVC), or polyether ether ketone (PEEK). The material may be loaded with a radiopaque material to aid in visualizing the dilator during a medical procedure.

In one example, the dilator tip 130 may be formed separately from the shaft portion 110 and attached to the distal end 112 of the shaft portion 110. The dilator tip 130 may be attached to the distal end 112 of the shaft portion 110 in any suitable manner. For example, the dilator tip 130 may be attached to the distal end 112 of the shaft portion 110 by an adhesive or a mechanical coupling mechanism (e.g., threads, sleeve, friction fit, or snap fit). In another example, the dilator tip 130 may be attached to the distal end 112 of the shaft portion 110 by heat bonding (e.g., by applying heat to the dilator tip and the shaft portion so that the materials flow and meld together).

In another example, the dilator tip 130 may be formed integrally with the shaft portion 110. For example, the shaft portion 110 may be formed, and the distal end 112 of the shaft portion may be introduced (e.g., pushed) into a tip mold to form the dilator tip 130. Upon introducing the shaft portion 110 into the tip mold, the distal end 112 of the shaft portion may be shaped (e.g., by heat and/or pressure) to form the dilator tip 130. In another example, the shaft portion 110 may be formed, and the distal end 112 of the shaft portion may be positioned within an insert mold. The material for the dilator tip 130 may be introduced into the insert mold around the distal end 112 of the shaft portion 110 to form the dilator tip at the distal end of the shaft portion. In any of these examples, the mold used to form the dilator tip 130 may include a surface with a pattern that is complementary to the textured outer surface 138 of the dilator tip. In other words, the mold may include surface features complementary to the surface features to be formed on the outer surface 138 of the dilator tip 130.

The dilator 100 may be used to enlarge an opening through a body tissue of a patient. In one example, the dilator 100 may be used to enlarge an opening through the skin of the patient as part of a percutaneous medical procedure. In other examples, the dilator 100 may be used to enlarge an opening through any other body tissue to gain access to any desired location within the patient's body. For example, the dilator 100 may be used to enlarge an opening through the wall of the stomach for placement of a gastric feeding tube, or G-tube. In another example, the dilator 100 may be used to enlarge an opening through the wall of the esophagus for placement of a tracheostomy tube for airway management.

A hollow needle may be inserted through the body tissue to create the opening in the body tissue. A guide wire may be inserted through the needle (e.g., through a lumen within the needle), and the needle may be withdrawn over the guide wire and removed, leaving the guide wire in place within the opening. The dilator 100 may be introduced over the guide wire. For example, a proximal end of the guide wire may be inserted into the distal end opening of the dilator tip 130 such that the guide wire is received within the lumen of the dilator 100. The dilator 100 may be advanced distally over the guide wire until the distal end 134 of the dilator tip 130 contacts the body tissue at the opening. The dilator 110 may be further advanced distally relative to the guide wire to push the dilator tip 130 into the opening in the body tissue.

Upon introducing the dilator tip 130 into the opening, a portion of the outer surface 138 of the dilator tip may engage the body tissue surrounding the opening. As the dilator is advanced farther into the opening, the portion of the outer surface 138 engaged with the body tissue surrounding the opening may have an increasingly larger diameter. In other words, as the dilator 110 is advanced into the opening, the body tissue surrounding the opening may be engaged by a first portion of the dilator tip 130 positioned near the distal end 134 of the dilator tip. As the dilator 110 is advanced farther into the opening, the body tissue surrounding the opening may be engaged by a second portion of the dilator tip 130 positioned proximal of the first portion and having a larger diameter than the first portion as described above. The larger diameter of the second portion of the dilator tip 130 may cause the size of the opening to increase to receive the second portion of the dilator tip. In this manner, the dilator 110 may be further advanced into the opening in the body tissue to enlarge the opening.

In one example, the dilator tip 130 may include one or more projections 140 as described above. During advancement of the dilator tip 130 through the opening in the body tissue, the projections 140 may engage the tissue surrounding the opening. For example, the dilator tip 130 may be advanced into the opening until a projection 140 positioned near the distal end 134 of the dilator tip 130 (e.g., the distal projection 144) engages the tissue surrounding the opening. The projection 140 may push a segment of the body tissue surrounding the opening outward away from the longitudinal axis of the dilator tip 130. As the dilator tip 130 is advanced farther into the opening, the projection 140 may pass through the opening, and the engaged segment of the body tissue may contract back toward the longitudinal axis of the dilator tip 130. In other words, as the projection 140 passes through the opening, the engaged segment of the body tissue may be pushed away from the outer surface 138 by the projection 140 and then allowed to retract back toward the outer surface once the projection has passed through the opening.

The body tissue may repeatedly be pushed away from the longitudinal axis of the dilator tip 130 and allowed to retract back toward the longitudinal axis of the dilator tip as successive projections 140 pass through the opening. The outward and inward motion of the body tissue surrounding the opening may reduce the friction between the body tissue and the outer surface 138 of the dilator tip 130. For example, the motion of the body tissue may reduce the tendency of the body tissue to stick to the outer surface 138 of the dilator tip 130. Additionally, or alternatively, the movement of the body tissue may create a massaging action that may progressively push different segments of the body tissue surrounding the opening out of the way of the outer surface 138 to enable the dilator tip 130 to pass through the opening with reduced resistance. The massaging motion may break tissue connections of the body tissue surrounding the opening, which may aid in further advancing the dilator tip 130 into the opening. Additionally, or alternatively, the outward movement of the body tissue away from the longitudinal axis of the dilator tip 130 may stretch the body tissue momentarily to prepare the body tissue for the larger diameter segment of the dilator tip positioned just proximal of the projection 140. In other words, the projection passing through the opening of the body tissue may prepare the body tissue to receive the next longitudinal portion of the dilator tip 130, which may have a greater diameter.

The dilator tip 130 may include a concave taper as described above. The concave taper may aid in advancing the dilator tip 130 through the opening. For example, the outer surface 138 of the dilator tip 130 may have a relatively flat taper near the distal end 134 and a relatively steep taper near the proximal end 132 as described above. The distance between the outer edge of a projection 140 positioned closer to the distal end 134 of the dilator tip 130 (e.g., the distal projection 144) and the portion of the outer surface 138 positioned just proximal of the projection may be greater than the distance between the outer edge of a projection positioned closer to the proximal end 132 of the dilator tip (e.g., the proximal projection 142) and the portion of the outer surface positioned just proximal of the projection. In other words, when the body tissue is engaged by a projection 140 positioned on the flatter portion of the dilator tip 130, the tissue may be allowed to retract a greater distance before contacting the outer surface 138 than when the body tissue is engaged by a projection positioned on the steeper portion of the dilator tip. The greater range of movement of the body tissue surrounding the opening during initial insertion of the dilator tip 130 into the opening may aid in initially stretching the body tissue and/or breaking tissue connections, as described above, which may aid in further advancing the dilator tip through the opening.

In another example, the dilator tip 130 may include one or more indentations 150 and/or grooves 160 as described above. The indentations 150 and/or grooves 160 may reduce the surface area of the outer surface 138 of the dilator tip 130 which may be in contact with the body tissue surrounding the opening. Such a reduced contact area may reduce the frictional force between the dilator tip 130 and the body tissue to reduce the amount of resistance to the dilator tip passing through the opening.

During advancement of the dilator tip 130 through the opening in the body tissue, the dilator 110 may be rotated about the longitudinal axis of the dilator. In other words, the dilator 110 may be twisted during advancement of the dilator tip 130 through the opening in the body tissue. Such a rotation may reduce the friction between the dilator tip 130 and the body tissue surrounding the opening to aid in advancing the dilator 110 farther into the opening. In one example, the dilator tip 130 may include surface features (e.g., projections 140, indentations 150, and/or grooves 160), which may be arranged in a spiral pattern as described above. Upon rotation of the dilator tip 130, the spiral pattern of the surface features may urge the dilator proximally or distally relative to the body tissue. For example, upon clockwise rotation (when viewed from the proximal end of the dilator 110) the surface features arranged in the right-handed spiral pattern may urge the dilator distally relative to the body tissue. In other words, the surface features arranged in the right-handed spiral pattern may engage the body tissue surrounding the opening (e.g., like the threads of a screw) to urge the dilator 110 longitudinally into the opening. In another example, upon counterclockwise rotation, the surface features arranged in the left-handed spiral pattern may urge the dilator 110 longitudinally into the opening. In yet another example, the surface features arranged in the double spiral pattern may urge the dilator longitudinally into or out of the opening upon clockwise and/or counterclockwise rotation.

In any of the examples described herein, the reduced friction between the dilator tip 130 and the body tissue surrounding the opening may reduce the resistance which may be experienced while advancing the dilator tip 130 through the opening. Such reduced resistance may reduce the pain and/or discomfort experienced by the patient during advancement of the dilator tip 130 through the opening. Additionally, or alternatively, the reduced friction between the dilator tip 130 and the body tissue surrounding the opening may enable a greater difference between the diameter of the dilator tip at the distal end 134 and the diameter of the dilator tip at the proximal end 132. This may enable the opening in the body tissue to be enlarged using the single dilator 110 as opposed to multiple, progressively larger dilators. The single dilator 110 may be capable of enlarging the opening by a greater amount than conventional dilators without excessive friction between the dilator tip 130 and the body tissue and without causing excessive pain and/or discomfort for the patient.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. A dilator for enlarging an opening through a body tissue, the dilator comprising:
a shaft portion extending along a longitudinal axis of the dilator and comprising a proximal end and a distal end; and
a dilator tip positioned at the distal end of the shaft portion and comprising a proximal end, a distal end, and an outer surface extending between the proximal end and the distal end of the dilator tip;
wherein the outer surface comprises a first diameter at the proximal end of the dilator tip and a second diameter at the distal end of the dilator tip, the first diameter is larger than the second diameter, and the outer surface comprises a concave taper between the first diameter and the second diameter; and
wherein the dilator tip comprises a plurality of projections extending away from the outer surface of the dilator tip.

2. The dilator of claim 1, wherein the projections are arranged in a plurality of rows spaced from one another about a circumference of the dilator tip, and each row extends longitudinally along the outer surface.

3. The dilator of claim 1, wherein the projections are arranged in a spiral pattern extending longitudinally and circumferentially about the dilator tip.

4. The dilator of claim 1, wherein the plurality of projections comprises a first series of projections arranged in a first spiral pattern extending longitudinally and circumferentially in a first circumferential direction about the dilator tip and a second series of projections arranged in a second spiral pattern extending longitudinally and circumferentially in a second circumferential direction opposite the first circumferential direction.

5. The dilator of claim 1, wherein the plurality of projections comprises a proximal projection positioned near the proximal end of the dilator tip and a distal projection positioned near the distal end of the dilator tip, and a size of the proximal projection is different than a size of the distal projection.

6. The dilator of claim 5, wherein the size of the proximal projection is larger than the size of the distal projection.

7. The dilator of claim 5, wherein the size of the proximal projection comprises a diameter and a height, and the size of the distal projection comprises a diameter and a height.

8. The dilator of claim 7, wherein the diameter of the proximal projection is greater than the diameter of the distal projection.

9. The dilator of claim 7, wherein the height of the proximal projection is greater than the height of the distal projection.

10. The dilator of claim 1, wherein the concave taper of the outer surface comprises a first taper near the proximal end of the dilator tip and a second taper near the distal end of the dilator tip, and the first taper is greater than the second taper.

11. The dilator of claim 10, wherein the first taper is between about 2.4° and about 3.6°, and the second taper is between about 1.2° and about 2.4°.

12. The dilator of claim 1, wherein the outer surface is curved inward toward the longitudinal axis of the dilator.

13. A dilator for enlarging an opening through a body tissue, the dilator comprising:
a shaft portion extending along a longitudinal axis of the dilator and comprising a proximal end and a distal end; and
a dilator tip positioned at the distal end of the shaft portion and comprising a proximal end, a distal end, and an outer surface extending between the proximal end and the distal end of the dilator tip and comprising a proximal region positioned near the proximal end of the dilator tip, a distal region positioned near the distal end of the dilator tip, and a diameter that decreases in a proximal to distal longitudinal direction;
wherein the proximal region of the outer surface comprises a taper that is greater than a taper of the distal region of the outer surface.

14. The dilator of claim 13, wherein the taper of the proximal region of the outer surface is between about 2.4° and about 3.6°, and the taper of the distal region of the outer surface is between about 1.2° and about 2.4°.

15. The dilator of claim 13, wherein the dilator tip comprises a plurality of projections extending away from the outer surface of the dilator tip.

16. The dilator of claim 15, wherein the plurality of projections comprises a proximal projection and a distal projection positioned distal of the proximal projection, and at least one of a diameter or a height of the proximal projection is greater than a respective diameter or height of the distal projection.

17. The dilator of claim 15, wherein the projections are arranged in a plurality of rows spaced from one another about a circumference of the dilator tip, and each row extends longitudinally along the outer surface.

18. The dilator of claim 15, wherein the projections are arranged in a spiral pattern extending longitudinally and circumferentially about the dilator tip.

19. A method of enlarging an opening in a body tissue, the method comprising:
introducing a distal end of a dilator tip into the opening, the dilator tip comprising an outer surface comprising an increasing diameter in a distal to proximal longitudinal direction, the diameter increasing in a concave taper;
advancing the dilator tip into the opening to enlarge the opening with the increasing diameter of the dilator tip; and
manipulating a portion of the body tissue surrounding the opening with a plurality of projections extending away from the outer surface of the dilator tip.

20. The method of claim 19, wherein the projections are arranged in a spiral pattern extending longitudinally and circumferentially about the dilator tip, and advancing the dilator tip into the opening comprises rotating the dilator tip about a longitudinal axis of the dilator tip such that the spiral pattern of projections urges the dilator tip into the opening.
